(19) United States
(12) Patent Application Publication
(10) Pub. No.: US 2017/0283764 A1
(43) Pub. Date: Oct. 5, 2017

(54) PROCESSING OF PLANT MATERIAL INTO BACTERIAL FEEDSTOCK

(71) Applicant: SHANGHAI ZHIYI INFORMATION TECHNOLOGY LTD, Shanghai (CN)

(72) Inventor: Feng Hong, Shanghai (CN)

(21) Appl. No.: 15/507,552

(22) PCT Filed: Aug. 29, 2014
(86) PCT No.: PCT/CN2014/085515

§ 371 Date: Feb. 28, 2017

(51) Int. Cl. C12N 1/22; C12P 19/04; C12P 19/14

(52) U.S. Cl. CPC C12N 1/22; C12P 19/14; C12P 19/04

(57) ABSTRACT

Methods and compositions relating to processing of plant material into bacterial feedstock, such as bacterial feedstock suitable for nanocellulose production, are disclosed. Cellulosic plant fiber may be contacted with a catalyst such as an acid catalyst or an enzymatic catalyst or both, and the mixture can be hydrolyzed into a hydrolysate. The hydrolysate may be used to form a culture medium which can be used to support bacterial growth to form the nanocellulose.

### FIELD

Disclosed are methods and compositions for processing materials such as plant materials into bacterial feedstock, such as for nanocellulose production.

### BACKGROUND

Bacterial nanocellulose (BNC) is an extracellular biopolymer produced in a microbial fermentation process. Vinegar bacteria are commonly used in the production of BNC. BNC has many excellent properties, such as a high purity (free of lignin and hemicellulose), a high crystallinity, a high degree of polymerization, a nano-structured network, a high wet tensile strength, a high water-holding capacity, and good biocompatibility. These characteristics distinguish it from plant cellulose. In view of these positive features, BNC is considered for applications in many different fields, such as biomedicine, food industry, cosmetics, advanced acoustic diaphragms, paper-making, and textile industry.

### SUMMARY

Some embodiments of the present disclosure relate to methods of making bacterial nanocellulose. In an embodiment, a method of making bacterial nanocellulose may include contacting a cellulosic plant fiber with a catalyst selected from an acid catalyst, an enzymatic catalyst or both, to form a reaction mixture; subjecting the reaction mixture to conditions sufficient to hydrolyze at least a portion of the cellulosic plant fiber into a hydrolysate; forming a culture medium with the hydrolysate; inoculating the culture medium with at least one bacteria; and incubating the culture medium and the bacteria under conditions sufficient to form the bacterial nanocellulose.

In an embodiment, a method of making a culture medium may include: contacting a cellulosic plant fiber with a catalyst selected from an acid catalyst, an enzymatic catalyst or both, to form a reaction mixture; subjecting the reaction mixture to conditions sufficient to hydrolyze at least a portion of the cellulosic plant fiber into a hydrolysate; and forming the culture medium with the hydrolysate.

In an embodiment, a method of preparing a bacterial system may include: contacting a cellulosic plant fiber with a catalyst selected from an acid catalyst, an enzymatic catalyst or both, to form a reaction mixture; subjecting the reaction mixture to conditions sufficient to hydrolyze at least a portion of the cellulosic plant fiber into a hydrolysate; forming a culture medium with the hydrolysate; and inoculating the culture medium with at least one bacteria to form the bacterial system.

In an embodiment, a culture medium may include a hydrolysate prepared by contacting a cellulosic plant fiber with a catalyst selected from an acid catalyst, an enzymatic catalyst or both, to form a reaction mixture; and subjecting the reaction mixture to conditions sufficient to hydrolyze at least a portion of the cellulosic plant fiber into the hydrolysate.

In an embodiment, a bacterial system may include: a culture medium including a hydrolysate prepared by contacting a cellulosic plant fiber with a catalyst selected from an acid catalyst, an enzymatic catalyst or both, to form a reaction mixture; and subjecting the reaction mixture to conditions sufficient to hydrolyze at least a portion of the cellulosic plant fiber into the hydrolysate; and at least one bacteria inoculated into the culture medium.

The foregoing summary is illustrative only and is not intended to be in any way limiting. In addition to the illustrative aspects, embodiments, and features described above, further aspects, embodiments, and features will become apparent by reference to the drawings and the following detailed description.

### DETAILED DESCRIPTION

Bacterial nanocellulose (BNC) can be a valuable product possessing excellent properties such as transparency, tensile strength, fiber-binding ability, biocompatibility, and biodegradability. However, the production of BNC can be expensive due to the high costs associated with the culture media. Production of BNC can also lead to relatively low-yields.

A number of methods have been proposed for the manufacture of BNC on an industrial scale and in commercially useful forms. However, for such methods to be commercially viable, a low-cost culture medium, and methods for the manufacture of such a medium, must be developed.

Carbon sources utilized in fermentation processes for BNC production include monosaccharides (such as glucose and fructose), disaccharides (such as sucrose and maltose), and alcohols (such as ethanol, glycerol, and mannitol). These feedstocks are usually expensive, and may sometimes result in low yields of BNC, which may then lead to high BNC production cost. The high production cost limits the scale of industrial manufacture of BNC and consequently becomes a bottleneck for extending the applications for BNC.

Some embodiments of the present disclosure relate to methods of making bacterial nanocellulose, for example from plant cellulosic material. The method may include contacting a cellulosic plant fiber with a catalyst selected from an acid catalyst, an enzymatic catalyst or both, to form a reaction mixture; subjecting the reaction mixture to conditions sufficient to hydrolyze at least a portion of the cellulosic plant fiber into a hydrolysate; forming a culture medium with the hydrolysate; inoculating the culture medium with at least one bacteria; and incubating the culture medium and the bacteria under conditions sufficient to form the bacterial nanocellulose. In some embodiments, the method of making bacterial nanocellulose may further include separating any unhydrolyzed cellulosic plant fiber from the hydrolysate. In some embodiments, the method of making bacterial nanocellulose may further include adding the unhydrolyzed cellulosic plant fiber to the reaction mixture before the subjecting step. In some embodiments, the separating step may include filtration, centrifugation, or both. In some embodiments, the method of making bacterial nanocellulose may further include fragmenting the cellulosic plant fiber before contacting with the catalyst. In some embodiments, the cellulosic plant fiber may have an average particle size of about 250 μm to about 420 μm in diameter. For example, the average particle size may be about 250 μm, 260 μm, 270 μm, 280 μm, 290 μm, 300 μm, 310 μm, 230 μm, 330 μm, 340 μm, 350 μm, 360 μm, 370 μm, 380 μm, 390 μm, 400 μm, 410 μm, 420 μm, or any size between these values.

In some embodiments, the method of making bacterial nanocellulose may further include crushing a cellulosic plant to form the cellulosic plant fiber and a juice before the fragmenting step. In some embodiments, the method of making bacterial nanocellulose may further include adding the juice to the hydrolysate when forming the culture medium.

In some embodiments, the cellulosic plant fiber can be Sorghum bagasse, sugar cane bagasse, or both. In some embodiments the cellulosic plant can be Sorghum, sugar cane, or both.

In some embodiments, forming the culture medium may include adding at least one nitrogen source, at least one trace element, or both to the hydrolysate. In some embodiments, the nitrogen source may include organic nitrogen. The organic nitrogen may for example be peptone, yeast extract, tryptone, or a combination thereof. The peptone, yeast extract and glucose may be present in concentrations such as (w/v) about 0.3% peptone, about 0.5% yeast extract, and about 2.5% glucose, though other concentrations are contemplated. In some embodiments, the nitrogen source may include inorganic nitrogen. The inorganic nitrogen may for example be ammonia sulfate, ammonia chloride, or both. In some embodiments, the nitrogen source can include both organic and inorganic nitrogen. In some embodiments, the nitrogen source may be present in the culture medium at a concentration of about 0.1% to about 1% by weight. For example, the concentration of the nitrogen source may be about 0.1%, about 0.2%, about 0.3%, about 0.4%, about 0.5%, about 0.6%, about 0.7%, about 0.8%, about 0.9%, about 1.0% by weight, or any concentration between these values.

In some embodiments, the hydrolysate may be present in the culture medium at a concentration of about 1% to about 10% by weight. For example, the concentration of the hydrolysate may be about 1%, about 2%, about 3%, about 4%, about 5%, about 6%, about 7%, about 8%, about 9%, about 10% by weight, or any concentration between these values. In some embodiments, the trace element may include calcium, magnesium, or both calcium and magnesium. In some embodiments, the trace element may be present in the culture medium at a concentration of about 0.1% to about 0.5% by weight. For example, the concentration of the trace element may be about 0.1%, about 0.2%, about 0.3%, about 0.4%, about 0.5% by weight, or any concentration between these values.

In some embodiments, the bacteria can be Gluconacetobacter xylinus, Gluconacetobacter hansenii, Gluconobacter oxydans, Rhizobium sp., Sarcina sp., Pseudomounas sp., Achromobacter sp., Alcaligenes sp., Aerobacter sp., Azotobacter sp., Agrobacterium sp., Seudomonas cepacia, Campylobacter jejuni, or a combination thereof. In some embodiments, the bacteria may be present in the culture medium at a concentration of about 3% to about 15% by volume. For example, the concentration of the bacteria may be about 3%, about 5%, about 7%, about 9%, about 11%, about 13%, about 15% by volume, or any concentration between these values.

In some embodiments, the hydrolysate may have a reducing sugar concentration of about 5 g/L to about 200 g/L. For example, the reducing sugar concentration can be about 5, about 6, about 7, about 8, about 9, about 10, about 20, about 30, about 40, about 50, about 60, about 70, about 80, about 90, about 100, about 110, about 120, about 130, about 140, about 150, about 160, about 170, about 180, about 190, about 195, about 200 g/L, or any concentration between these values.

In some embodiments, the incubating step in the method of making bacterial nanocellulose may include sterilizing media, inoculating a bacterial culture into the media and incubating the culture to cause production of bacterial nanocellulose. In several embodiments, the bacteria used to produce nanocellulose may be inoculated into the culture medium after the medium has been autoclaved, for example at temperatures between about 105° C. and 121° C. for times ranging between about 15 to about 30 minutes. For example, the medium may be autoclaved at temperatures between about 105° C. to about 110° C., about 110° C. to about 115° C., about 115° C. to about 120° C., about 120° C. to about 125° C., or any temperatures between these autoclave temperatures. Also, for example, autoclave times may range from about 15 minutes to about 17 minutes, about 17 minutes to about 19 minutes, about 19 minutes to about 21 minutes, about 21 minutes to about 23 minutes, about 23 minutes to about 25 minutes, about 25 minutes to about 27 minutes, about 27 minutes to about 30 minutes, or any times between these autoclave times. Additionally (or in some embodiments, in place of autoclaving), the medium may be sterilized by filtration with sterile filters. The bacteria are inoculated into the sterile medium until the bacteria are at a concentration of between about 5% and about 10% (volume/volume). For example, the bacteria can be inoculated to a concentration of about 5% to about 6%, about 6% to about 7%, about 7% to about 8%, about 8% to about 9%, about 9% to about 10%, or any concentration between these concentrations. After inoculation, the culture is incubated at temperatures between about 20° C. and about 37° C. For example, the temperature can be about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C. or any temperature between these values. In some embodiments, the incubating step may include incubating the culture medium and the bacteria for about 1 day to about 30 days. For example, the incubating can be performed for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or any length of time between these values. In some embodiments, the incubating step may include incubating the culture medium and the bacteria in a shaking incubator or a static incubator. In some embodiments, the incubating step may include incubating the culture medium and the bacteria in a shaking incubator that rotates at a speed of about 5 rpm to about 300 rpm. For example, the rotation can be at a speed of about 5 rpm, about 6 rpm, about 7 rpm, about 8 rpm, about 9 rpm, about 10 rpm, about 20 rpm, about 30 rpm, about 40 rpm, about 50 rpm, about 60 rpm, about 70 rpm, about 80 rpm, about 90 rpm, about 100 rpm, about 110 rpm, about 120 rpm, about 130 rpm, about 140 rpm, about 150 rpm, about 160 rpm, about 170 rpm, about 180 rpm, about 190 rpm, about 200 rpm, about 210 rpm, about 220 rpm, about 230 rpm, about 240 rpm, about 250 rpm, about 260 rpm, about 270 rpm, about 280 rpm, about 290 rpm, about 300 rpm, or any speed between these values.

In some embodiments, the method of making bacterial nanocellulose may further include harvesting the bacterial nanocellulose from the culture medium. In some embodiments, after the harvesting, the method may further include contacting the bacterial nanocellulose with a base under conditions to remove residual bacteria and culture medium. The base may, for example, be NaOH, KOH, NH₄OH, or a combination thereof. In some embodiments, contacting the bacterial nanocellulose with the base may include heating at about 70° C. to about 120° C. For example, the heating may be carried out at about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., about 105° C., about 110° C., about 115° C., about 120° C., or any temperature between these values. In some embodiments, contacting the bacterial nanocellulose with the base may include heating for about 90 minutes to about 150 minutes. For example, the heating may be carried out for about 90 minutes, 91 minutes, 92 minutes, 93 minutes, 94 minutes, 95 minutes, 96 minutes, 97 minutes, 98 minutes, 99 minutes, 100 minutes, 101 minutes, 102 minutes, 103 minutes, 104 minutes, 105 minutes, 106 minutes, 107 minutes, 108 minutes, 109 minutes, 110 minutes, 111 minutes, 112 minutes, 113 minutes, 114 minutes, 115 minutes, 116 minutes, 117 minutes, 118 minutes, 119 minutes, 120 minutes, 121 minutes, 122 minutes, 123 minutes, 124 minutes, 125 minutes, 126 minutes, 127 minutes, 128 minutes, 129 minutes, 130 minutes, 131 minutes, 132 minutes, 133 minutes, 134 minutes, 135 minutes, 136 minutes, 137 minutes, 138 minutes, 139 minutes, 140 minutes, 141 minutes, 142 minutes, 143 minutes, 144 minutes, 145 minutes, 146 minutes, 147 minutes, 148 minutes, 149 minutes, 150 minutes, or any length of time between these values. In some embodiments, the base may be in aqueous form having a concentration of about 0.5% to about 8% by weight. For example, the base may have a concentration of about 0.5%, about 0.6%, about 0.7%, about 0.8%, about 0.9%. about 1.0%, about 2%, about 3%, about 4%, about 5%, about 6%, about 7%, about 8%, about 9%, or any concentration between these values.

In some embodiments, the subjecting step in the method of making bacterial nanocellulose may include subjecting the reaction mixture to a temperature of about 25° C. to about 200° C. For example, the reaction mixture may be subject to temperatures of about 25° C. to about 30° C., about 30° C. to about 40° C., about 40° C. to about 50° C., about 50° C. to about 60° C., about 60° C. to about 70° C., about 70° C. to about 80° C., about 80° C. to about 90° C., about 90° C. to about 100° C., about 100° C. to about 110° C., about 110° C. to about 120° C., about 120° C. to about 130° C., about 130° C. to about 140° C., about 140° C. to about 150° C., about 150° C. to about 160° C., about 160° C. to about 170° C., about 170° C. to about 180° C., about 180° C. to about 190° C., about 190° C. to about 200° C., or any temperature in between these values. In some embodiments, the subjecting step may occur for about 10 minutes to about 48 hours. For example, the subjecting stem may be about 10 minutes to about 15 minutes, about 15 minutes to about 20 minutes, about 20 minutes to about 25 minutes, about 25 minutes to about 30 minutes, about 30 minutes to about 1 hour, about 1 hour to about 4 hours, about 4 hours to about 8 hours, about 8 hours to about 12 hours, about 12 hours to about 16 hours, about 16 hours to about 20 hours, about 20 hours to about 24 hours, about 24 hours to about 28 hours, about 28 hours to about 32 hours, about 32 hours to about 36 hours, about 36 hours to about 40 hours, about 40 hours to about 44 hours, about 44 hours to about 48 hours, and any time between those values.

In some embodiments, contacting the cellulosic plant fiber with the catalyst may include contacting an aqueous form of the catalyst with the cellulosic plant fiber. In some embodiments, the cellulosic plant fiber and the aqueous form of the catalyst may be present in the reaction mixture at a ratio of about 1:5 to about 1:30 by weight to volume (w/v). For example, the ratio can be about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, or any ratio between these values.

In some embodiments, the catalyst can be an acid catalyst. For example, the acid catalyst may be an inorganic acid. For example, the acid catalyst may comprise H₂SO₄, HCl, H₃PO₄, HNO₃, or combinations thereof. The acid catalyst may also be an organic acid. For example, organic acids such as acetic acid, citric acid, phytic acid, heteropolyacid, or a combination thereof may be used. In some embodiments, the acid catalyst may be an aqueous acid solution having a concentration of about 0.3% to about 10% w/v. For example, the aqueous acid solution may have a concentration of about 0.3% to about 0.5%, about 0.5% to about 0.7%, about 0.7% to about 1.0%, about 1.0% to about 2.0%, about 2.0% to about 3.0%, about 3.0% to about 4.0%, about 4.0% to about 5.0%, about 5.0% to about 6.0%, about 6.0% to about 7.0%, about 7.0% to about 8.0%, about 8.0% to about 9.0%, about 9.0% to about 10.0% and any concentration between those concentrations listed. In some embodiments, where the catalyst is an acid catalyst, contacting the cellulosic plant fiber with the catalyst may include contacting for about 12 hours to about 24 hours, for example, 12 hours, 13 hours, 14 hours, 15 hours, 16 hours, 17 hours, 18 hours, 19 hours, 20 hours, 21 hours, 22 hours, 23 hours, 24 hours or any length of time between these values. In some embodiments, where the catalyst is an acid catalyst, the subjecting step may include subjecting the reaction mixture to a temperature of about 25° C. to about 200° C. For example, the subject stem may include subjecting the reaction mixture to a temperature of about 25° C. to about 30° C., about 30° C. to about 40° C., about 40° C. to about 50° C., about 50° C. to about 60° C., about 60° C. to about 70° C., about 70° C. to about 80° C., about 80° C. to about 90° C., about 90° C. to about 100° C., about 100° C. to about 110° C., about 110° C. to about 120° C., about 120° C. to about 130° C., about 130° C. to about 140° C., about 140° C. to about 150° C., about 150° C. to about 160° C., about 160° C. to about 170° C., about 170° C. to about 180° C., about 180° C. to about 190° C., about 190° C. to about 200° C., or any temperature in between these values. In some embodiments, where the catalyst is an acid catalyst, the subjecting step may occur for about 10 minutes to about 180 minutes. For example, in some embodiments, the subjecting step may occur for about 10 minutes to about 15 minutes, about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, about 55 minutes to about 60 minutes, about 60 minutes to about 65 minutes, about 65 minutes to about 70 minutes, about 70 minutes to about 75 minutes, about 75 minutes to about 80 minutes, about 80 minutes to about 85 minutes, about 85 minutes to about 90 minutes, about 90 minutes to about 95 minutes, about 95 minutes to about 100 minutes, about 100 minutes to about 110 minutes, about 110 minutes to about 120 minutes, about 120 minutes to about 130 minutes, about 130 minutes to about 140 minutes, about 140 minutes to about 150 minutes, about 150 minutes to about 160 minutes, about 160 minutes to about 170 minutes, about 170 minutes to about 180 minutes, or any time period between those listed.

According to certain embodiments, the times and temperatures for a hydrolysis reactions are inversely correlated. For example, in those embodiments in which a higher temperature is used, the time for the progression of the hydrolysis reaction may be reduced. Determination of the precise times and temperatures are readily made without undue experimentation.

In some embodiments, the catalyst may be an enzymatic catalyst. In some embodiments, the enzyme may be a saccharification enzyme. For example, the saccharification enzyme can be a cellulase, hemicellulose, xylanase, endogluconase, cellobiase, protease, lipase, amylase, glucan glucohydrolase, glucoamylase, or a combination thereof. In some embodiments, the enzymatic catalyst may have an enzyme unit of about 1 U to about 700 U. For example, the enzymatic catalyst may have a unit concentration of about 1 U to about 50 U, about 50 U to about 100 U, about 100 U to about 150 U, about 150 U to about 200 U, about 200 U to about 250 U, about 250 U to about 300 U, about 300 U to about 350 U, about 400 U to about 450 U, about 450 U to about 500 U, about 500 U to about 550 U, about 550 U to about 600 U, about 600 U to about 650 U, about 650 U to about 700 U, or any number of enzyme units between the unit values listed. In some embodiments, where the catalyst is an enzymatic catalyst, the subjecting step may occur for about 30 minutes to about 72 hours. For example, the subjecting step may be for about 30 minutes to about 1 hour, about 1 hour to about 4 hours, about 4 hours to about 8 hours, about 8 hours to about 12 hours, about 12 hours to about 16 hours, about 16 hours to about 20 hours, about 20 hours to about 24 hours, about 24 hours to about 28 hours, about 28 hours to about 32 hours, about 32 hours to about 36 hours, about 36 hours to about 40 hours, about 40 hours to about 44 hours, about 44 hours to about 48 hours, about 48 hours to 52 hours, about 52 hours to 56 hours, about 56 hours to 60 hours, about 60 hours to 64 hours, about 64 hours to 68 hours, about 68 hours to 72 hours, and any time between those values. In some embodiments, the subjecting step may include subjecting the reaction mixture to a temperature of about 25° C. to about 90° C. For example, the reaction mixture may be subjected to temperatures of about 25° C. to about 30° C., about 30° C. to about 35° C., about 35° C. to about 40° C., about 40° C. to about 45° C., about 45° C. to about 50° C., about 50° C. to about 55° C., about 55° C. to about 60° C., about 60° C. to about 65° C., about 65° C. to about 70° C., about 70° C. to about 75° C., about 75° C. to about 80° C., about 80° C. to about 85° C., about 85° C. to about 90° C. or temperature values between any of these temperatures. According to certain embodiments, the times and temperatures for a hydrolysis reactions are inversely correlated. For example, in those embodiments in which a higher temperature is used, the time for the progression of the hydrolysis reaction may be reduced. Similarly, alterations in the changes in the amount of enzyme used can result in changes in the time and/or the temperature required (for example, increased amounts of enzymes can achieve hydrolysis in reduced amounts of time). For example, Determination of the precise times and temperatures are readily made without undue experimentation.

In some embodiments, where the catalyst is an acid catalyst, the method of making bacterial nanocellulose may further include detoxifying the hydrolysate after the subjecting step, and before inoculating the culture medium with the at least one bacteria. In some embodiments, the detoxifying step may include adjusting a pH value of the hydrolysate to an alkaline pH with a base; incubating the hydrolysate; adjusting the pH value of the hydrolysate to an acidic pH with an acid; contacting the hydrolysate with activated carbon; and separating the activated carbon from the hydrolysate. In some embodiments, the detoxifying step may further include adjusting the pH of the hydrolysate to the acidic pH after separating the activated carbon from the hydrolysate.

In some embodiments, the base used in the detoxifying step can be NaOH, Ca(OH)₂, KOH, NH₄OH, or a combination thereof. In some embodiments, the alkaline pH may be about pH 10. For example, the alkaline pH may be about 9.9, about 9.95, about 9.99, about 10, about 10.01, about 10.05, about 10.1, or pH values between the values listed above. In some embodiments, the alkaline pH is pH 10.

In some embodiments, the acid used in the detoxifying step may be H₂SO₄, HCl, HNO₃, H₃PO₄, acetic acid, citric acid or a combination thereof. In some embodiments, the acidic pH may be about pH 5. For example, the acidic pH may be about 4.5, about 4.6, about 4.7, about 4.8, about 4.9, about 5, about 5.1, about 5.2, about 5.3, about 5.4, about 5.5, and any pH values between these values. In some embodiments, the acidic pH is pH 5.

In some embodiments, incubating the hydrolysate may include incubating at a temperature of about 20° C. to about 50° C. For example, the incubation of the hydrolysate may be at a temperature of between about 20° C. to about 25° C., about 25° C. to about 30° C., about 30° C. to about 35° C., about 35° C. to about 40° C., about 40° C. to about 45° C., about 45° C. to about 50° C., or any temperature between the temperatures listed. In some embodiments, incubating the hydrolysate may occur for about 6 hours to about 24 hours. For example, the hydrolysate may be incubated for about 6 hours to about 8 hours, about 8 hours to about 10 hours, about 10 hours to about 12 hours, about 12 hours to about 14 hours, about 14 hours to about 16 hours, about 16 hours to about 18 hours, about 18 hours to about 20 hours, about 20 hours to about 22 hours, about 22 hours to about 24 hours, or any time between these incubation times.

In some embodiments, contacting the hydrolysate with the activated carbon in the detoxifying step may include mixing the activated carbon with the hydrolysate for about 5 minutes to about 15 minutes. For example, the activated carbon is mixed with the hydrolysate for about 5 minutes to about 6 minutes, about 6 minutes to about 7 minutes, about 7 minutes to about 8 minutes, about 8 minutes to about 9 minutes, about 9 minutes to about 10 minutes, about 10 minutes to about 11 minutes, about 11 minutes to about 12 minutes, about 12 minutes to about 13 minutes, about 13 minutes to about 14 minutes, about 14 minutes to about 15 minutes, or any time between these times. In some embodiments, the activated carbon may be present in the hydrolysate at a concentration of about 1% w/v to about 30% w/v. For example, the activated carbon may be present in the hydrolysate at a concentration (weight/volume) of about 1% to about 3%, of about 3% to about 6%, of about 6% to about 10%, of about 10% to about 15%, of about 15% to about 20%, of about 20% to about 25%, of about 25% to about 30% or any concentration between the listed concentrations. In some embodiments, separating the activated carbon from the hydrolysate may include filtration, centrifugation, or both.

In an alternative embodiment of detoxifying the hydrolysate where the catalyst is an acid catalyst, the detoxifying of the hydrolysate may include adjusting a pH value of the hydrolysate to an alkaline pH with a base, or to an acidic pH with an acid; contacting the hydrolysate with an enzyme; and incubating the hydrolysate. In some embodiments, the detoxifying step may further include adjusting the pH value of the hydrolysate to the alkaline pH or to the acidic pH after incubating the hydrolysate. In some embodiments, the base used in the detoxifying step may be NaOH, Ca(OH)₂, KOH, NH₄OH, or a combination thereof. In some embodiments, the acid used in the detoxifying step may be H₂SO₄, HCl, HNO₃, H₃PO₄, acetic acid, citric acid or a combination thereof. In some embodiments, the alkaline pH may be about pH 7 to about pH 10. For example, the alkaline pH may be about pH 7, about pH 7.5, about pH 8, about pH 8.5, about pH 9, about pH 9.5, about pH 10, or any pH value between the listed values. In some embodiments, the acidic pH may be about pH 2 to about pH 5. For example, the acidic pH may be about pH 2, about pH 2.5, about pH 3, about pH 3.5, about pH 4, about pH 4.5, about pH 5, or any acidic pH between these pH values.

In some embodiments, the enzyme used in the detoxifying step may be laccase, or a peroxidase with hydrogen peroxide. In some embodiments, the enzyme may have an enzyme unit concentration of about 1 U/ml to about 50 U/ml. For example, the enzyme used in the detoxifying step may have an enzyme unit concentration of about 1 U/ml to about 5 U/ml, about 5 U/ml to about 10 U/ml, about 10 U/ml to about 15 U/ml, about 15 U/ml to about 20 U/ml, about 20 U/ml to about 25 U/ml, about 25 U/ml to about 30 U/ml, about 30 U/ml to about 35 U/ml, about 35 U/ml to about 40 U/ml, about 40 U/ml to about 45 U/ml, about 45 U/ml to about 50 U/ml, or any unit enzyme concentration between those listed. In one embodiment, the enzyme unit concentration is, for example, 2.75 U/mL. In some embodiments, the enzyme may be present in the hydrolysate at a concentration of about 1% to about 20% by volume. For example, the enzyme may be present in the hydrolysate at a concentration of about 1% by volume to about 3% by volume, about 3% by volume to about 6% by volume, about 6% by volume to about 9% by volume, about 9% by volume to about 12% by volume, about 12% by volume to about 15% by volume, about 15% by volume to about 18% by volume, about 18% by volume to about 20% by volume, or any percentage by volume between the amounts listed above.

In some embodiments, incubating the hydrolysate in the detoxifying step may include incubating at a temperature of about 20° C. to 90° C. For example, the hydrolysate may be incubated in the detoxifying step at temperatures between about 20° C. to about 25° C., about 25° C. to about 30° C., about 30° C. to about 35° C., about 35° C. to about 40° C., about 40° C. to about 45° C., about 45° C. to about 50° C., about 50° C. to about 55° C., about 55° C. to about 60° C., about 60° C. to about 65° C., about 65° C. to about 70° C., about 70° C. to about 75° C., about 75° C. to about 80° C., about 85° C. to about 85° C., about 85° C. to about 90° C., or any temperature between those temperatures listed. In some aspects, incubating the hydrolysate comprises incubating at a temperature of about 20° C. to 50° C. (e.g., about 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55° C.). In some embodiments, incubating the hydrolysate may include incubating at a temperature of about 20° C. to 50° C. In some embodiments, such as when an extremophilic enzyme is used, incubating the hydrolysate may include incubating at a temperature of about 60° C. to 90° C. In some embodiments, the incubation may occur for about 1 hour to about 48 hours. For example, the incubation may occur for about 1 hour to about 4 hours, about 4 hours to about 8 hours, about 8 hours to about 12 hours, about 12 hours to about 16 hours, about 16 hours to about 20 hours, about 20 hours to about 24 hours, about 24 hours to about 28 hours, about 28 hours to about 32 hours, about 32 hours to about 36 hours, about 36 hours to about 40 hours, about 40 hours to about 44 hours, about 44 hours to about 48 hours, or any incubation time between those values.

Some embodiments of the present disclosure relate to methods of making a culture medium. The method may include contacting a cellulosic plant fiber with a catalyst selected from an acid catalyst, an enzymatic catalyst or both, to form a reaction mixture; subjecting the reaction mixture to conditions sufficient to hydrolyze at least a portion of the cellulosic plant fiber into a hydrolysate; and forming the culture medium with the hydrolysate.

In some embodiments, forming the culture medium may include adding at least one nitrogen source, at least one trace element, or both to the hydrolysate. In some embodiments, the cellulosic plant fiber may be fragmented before contacting with the catalyst. In some embodiments, the cellulosic plant may be crushed to form the cellulosic plant fiber and a juice, before the fragmenting step. In some embodiments, the juice may be added to the hydrolysate when forming the culture medium. In some embodiments, the cellulosic plant fiber may have an average particle size of about 250 μm to about 420 μm in diameter, or an average particle size as described above.

In some embodiments, the cellulosic plant fiber can be Sorghum bagasse, sugar cane bagasse, or both. In some embodiments, the cellulosic plant can be Sorghum, sugar cane, or both. In one embodiment in which the cellulosic plant is sorghum, the method includes one or more of the following steps: (1) sweet sorghum stalks are crushed first to get sweet juice and sorghum bagasse; (2) the sorghum bagasse is ground to small pieces or powder; (3) the small pieces or powder are hydrolyzed by catalysts in a reaction container; the residual slag of the reaction mixture and the hydrolysate are separated by sucking filtration and/or centrifugation (the filtrate or supernatant after separation may be collected); (4) the residual slag are hydrolyzed again and then all hydrolysates are pooled together and refrigerated for spare; (5) the sweet juice or the hydrolysate is supplemented respectively with nitrogen sources and trace elements to prepare cultural media for fermentation; (6) manufacturing bacteria are inoculated into the culture medium and cultivated at 20-37° C. and 5 to 300 rpm or cultivated statically in an incubator at 20-37° C. for 3-12 days, and then the bacterial nanocellulose is harvested. The technology with sweet sorghum as feedstock of culture medium provides a new approach to manufacture bacterial nanocellulose with low production price, which can be used for industrial scale production.

In several embodiments, wherein the sorghum bagasse is ground to small pieces or powder, the resultant small pieces or powder may be hydrolyzed by catalysts in a reaction container. In several embodiments, hydrolytic catalysts may be mixed with the ground bagasse before hydrolysis. The hydrolytic catalysts may be acids or enzymes, depending on the embodiment. The mixture reacts at temperatures ranges from about 25 to about 200° C., such as for example, the reaction temperature ranges discussed above.

In several embodiments, the concentration of total reducing sugar in the filtrate or supernatant may be assayed. In some embodiments, the hydrolysate may have a reducing sugar concentration of about 5 g/L to about 200 g/L, or as described above. For example, the reducing sugar concentration may be about 5 g/L to about 20 g/L, 5 g/L to about 20 g/L, 5 g/L to about 20 g/L, 5 g/L to about 20 g/L, 5 g/L to about 20 g/L, 5 g/L to about 20 g/L, 5 g/L to about 20 g/L, 5 g/L to about 20 g/L, 5 g/L to about 20 g/L, 5 g/L to about 20 g/L, 5 g/L to about 20 g/L, 5 g/L to about 20 g/L, 5 g/L to about 20 g/L, or any reducing sugar concentration between those concentrations listed.

In some embodiments, the subjecting step may include subjecting the reaction mixture to a temperature of about 25° C. to about 200° C. for about 10 minutes to about 48 hours, or as described above. In some embodiments, contacting the cellulosic plant fiber with the catalyst may include contacting an aqueous form of the catalyst with the cellulosic plant fiber. In some embodiments, the cellulosic plant fiber and the aqueous form of the catalyst may be present in the reaction mixture at a ratio of about 1:5 to about 1:30 by weight to volume (w/v), or as described above.

In some embodiments of the method of making the culture medium, the catalyst may be an acid catalyst. In some embodiments, the acid catalyst may be H₂SO₄, HCl, H₃PO₄, HNO₃, acetic acid, citric acid, phytic acid, heteropolyacid or a combination thereof. In some embodiments, the acid catalyst may be an aqueous acid solution having a concentration of about 0.3% w/v to about 10% w/v, or as described above. In some embodiments, where the catalyst is an acid catalyst, contacting the cellulosic plant fiber with the catalyst may include contacting for about 12 hours to about 24 hours, or as described above. In some embodiments, where the catalyst is an acid catalyst, the subjecting step may include subjecting the reaction mixture to a temperature of about 25° C. to about 200° C., or as described above. In some embodiments, the subjecting step may occur for about 10 minutes to about 90 minutes, or as described above.

In some embodiments of the method of making the culture medium, the catalyst may be an enzymatic catalyst. In some embodiments, the enzyme can be a saccharification enzyme. The saccharification enzyme can be cellulase, hemicellulose, xylanase, protease, lipase, amylase, glucan glucohydrolase, glucoamylase, and a combination thereof. In some embodiments, the enzyme may have an enzyme unit of about 1 U to about 700 U, or as described above. In some embodiments, where the catalyst is an enzymatic catalyst, the subjecting step may occur for about 30 minutes to about 48 hours, or as described above. In some embodiments, where the catalyst is an enzymatic catalyst, the subjecting step may include subjecting the reaction mixture to a temperature of about 25° C. to about 90° C., or as described above.

In some embodiments, where the catalyst is an acid catalyst, the method of making the culture medium may further include detoxifying the hydrolysate after the subjecting step. In some embodiments, the detoxifying may include adjusting a pH value of the hydrolysate to an alkaline pH with a base; incubating the hydrolysate; adjusting the pH value of the hydrolysate to an acidic pH with an acid; contacting the hydrolysate with activated carbon;

and separating the activated carbon from the hydrolysate. In some embodiments, the detoxifying may further include adjusting the pH of the hydrolysate to the acidic pH after separating the activated carbon from the hydrolysate. In some embodiments, the alkaline pH may be about 10, or as described above. In some embodiments, the acidic pH may be about pH 5, or as described above. In some embodiments, incubating the hydrolysate may include incubating for about 6 hours to about 24 hours, or as described above, though other time periods are contemplated. In some embodiments, incubating the hydrolysate may include incubating at a temperature of about 20° C. to about 50° C., or as described above.

In some embodiments, contacting the hydrolysate with activated carbon may include mixing the activated carbon with the hydrolysate. In some embodiments, the mixing can occur for about 5 minutes to about 15 minutes, or as described above. In some embodiments, the activated carbon may be present in the hydrolysate at a concentration of about 1% w/v to about 30% w/v, or as described above.

In an alternative embodiment of detoxifying the hydrolysate, where the catalyst is an acid catalyst, the detoxifying step may include adjusting a pH value of the hydrolysate to an alkaline pH with a base or to an acidic pH with an acid; contacting the hydrolysate with an enzyme; and incubating the hydrolysate. In some embodiments, the detoxifying step may further include adjusting the pH value of the hydrolysate to the alkaline pH or to the acidic pH after incubating the hydrolysate. In some embodiments, the alkaline pH may be about pH 7 to about pH 10, or as described above. In some embodiments, the acidic pH may be about pH 2 to about pH 5, or as described above. In some embodiments, the selection of the pH value may depend on the optimum pH of the enzyme. If the enzyme is acidic, the pH value should be in the acidic range. If the enzyme is alkaline, the pH value should be in the alkaline range.

In some embodiments, the enzyme used in the detoxifying step may be laccase. In some embodiments, the enzyme may be peroxidase supplied with H₂O₂. In some embodiments, the enzyme may have an enzyme unit concentration of about 1-50 U/ml, or as described above. In some embodiments, the enzyme may be present in the hydrolysate at a concentration of about 1-20% by volume, or as described above. In some embodiments, incubating the hydrolysate in the detoxifying step may include incubating at a temperature of about 20° C. to 90° C., or as described above. In some embodiments, the enzyme may be an extremophile enzyme. In some embodiments, the incubating may occur for about 1 hour to about 48 hours, or as described above.

Some embodiments of the present disclosure relate to methods of preparing a bacterial system. The method may include contacting a cellulosic plant fiber with a catalyst selected from an acid catalyst, an enzymatic catalyst or both, to form a reaction mixture; subjecting the reaction mixture to conditions sufficient to hydrolyze at least a portion of the cellulosic plant fiber into a hydrolysate; forming a culture medium with the hydrolysate; and inoculating the culture medium with at least one bacteria to form the bacterial system.

In some embodiments, forming the culture medium may include adding at least one nitrogen source, at least one trace element, or both to the hydrolysate when forming the culture medium. In some embodiments, the cellulosic plant fiber may be fragmented before contacting with the catalyst. In some embodiments, a cellulosic plant may be crushed to form the cellulosic plant fiber and a juice, before the fragmenting step. In some embodiments, the juice may be added to the hydrolysate when forming the culture medium. In some embodiments, the cellulosic plant fiber may be Sorghum bagasse, sugar cane bagasse, or both. In some embodiments, the cellulosic plant may be Sorghum, sugar cane, or both.

In some embodiments, the bacteria may be Gluconacetobacter xylinus, Gluconacetobacter hansenii, Gluconobacter oxydans, Rhizobium sp., Sarcina sp., Pseudomounas sp., Achromobacter sp., Alcaligenes sp., Aerobacter sp., Azotobacter sp., Agrobacterium sp., Seudomonas cepacia, Campylobacter jejuni, or a combination thereof.

In some embodiments, the subjecting step may include subjecting the reaction mixture to a temperature of about 25° C. to about 200° C., or as described above. In some embodiments, the subjecting step may include incubating the reaction mixture for about 10 minutes to about 48 hours, or as described above. In some embodiments, the contacting step may include contacting an aqueous form of the catalyst with the cellulosic plant fiber.

In some embodiments, a culture medium may include a hydrolysate prepared by contacting a cellulosic plant fiber with a catalyst selected from an acid catalyst, an enzymatic catalyst or both, to form a reaction mixture; and subjecting the reaction mixture to conditions sufficient to hydrolyze at least a portion of the cellulosic plant fiber into the hydrolysate.

In some embodiments, the culture medium may include least at least one nitrogen source, at least one trace element, or both. In some embodiments, the cellulosic plant fiber can be Sorghum bagasse, sugar cane bagasse, or both. In some embodiments, the nitrogen source may include organic nitrogen, such as peptone, yeast extract, tryptone, or a combination thereof. In some embodiments, the nitrogen source may include inorganic nitrogen, such as ammonia sulfate, ammonia chloride, or both. In some embodiments, the nitrogen source may include both organic and inorganic nitrogen.

In some embodiments, the trace element may include calcium, magnesium or both. In some embodiments, the nitrogen source may be present in the culture medium at a concentration of about 0.1% to about 1% by weight or as described above, and the hydrolysate may be present in the culture medium at a concentration of about 1% to about 10% by weight or as described above, and the trace element may be present in the culture medium at a concentration of about 0.1% to about 0.5% by weight or as described above.

In some embodiments, the hydrolysate may have a reducing sugar concentration of about 5 g/L to about 200 g/L or as described above.

In some embodiments, a bacterial system may include a culture medium having a hydrolysate prepared by contacting a cellulosic plant fiber with a catalyst selected from an acid catalyst, an enzymatic catalyst or both, to form a reaction mixture; and subjecting the reaction mixture to conditions sufficient to hydrolyze at least a portion of the cellulosic plant fiber into the hydrolysate; and at least one bacteria inoculated into the culture medium. In some embodiments, the culture medium may further include at least one nitrogen source, at least one trace element, or both. In some embodiments, the nitrogen source may include organic nitrogen such as peptone, yeast extract, tryptone, or a combination thereof, or inorganic nitrogen such as ammonia sulfate, ammonia chloride, or both, or both organic and inorganic nitrogen.

In some embodiments, the bacteria may include Gluconacetobacter xylinus, Gluconacetobacter hansenii, Gluconobacter oxydans, Rhizobium sp., Sarcina sp., Pseudomounas sp., Achromobacter sp., Alcaligenes sp., Aerobacter sp., Azotobacter sp., Agrobacterium sp., Seudomonas cepacia, Campylobacter jejuni, or a combination thereof.

In some embodiments, the cellulosic plant fiber may be Sorghum bagasse, sugar cane bagasse, or both. In some embodiments, the trace element may include calcium or magnesium or both calcium and magnesium. In some embodiments, the hydrolysate may have a reducing sugar concentration of about 5 g/L to about 200 g/L or as described above.

The acid hydrolysate may be detoxified in some embodiments (see e.g., Table 1).

| Method | Description of Detoxification Procedure |
|---|---|
| A1 | Firstly, adjusting the pH value of the hydrolysate to 10.0 by NaOH and |
| | incubating it at 30° C. for 12 h followed by adjusting the pH value to 5.0 again. |
| | Secondly, adding 1-6% (w/v) activated charcoal into the hydrolysate and |
| | mixing them for 5-10 minutes. Finally, removing the activated charcoal from |
| | the hydrolysate and adjusting the pH value to 5.0 again. |
| B1 | Firstly, adjusting the pH value of the hydrolysate to 10.0 by Ca(OH)₂ and |
| | incubating it at 30° C. for 12 h followed by adjusting pH value to 5.0 again. |
| | Secondly, adding 2% (w/v) activated charcoal into the hydrolysate and mixing |
| | them for 5 minutes. Finally, removing the activated charcoal from the |
| | hydrolysate and adjusting the pH value to 5.0 again. |
| C1 | Firstly, adjusting the pH value of the hydrolysate to 10.0 by ammonia and |
| | incubating it at 30° C. for 12 h followed by adjusting the pH value to 5.0 again. |
| | Secondly, adding 2% (w/v) activated charcoal into the hydrolysate and mixing |
| | them for 5 minutes. Finally, removing the activated charcoal from the |
| | hydrolysate and adjusting the pH value to 5.0 again. |
| A2 | Firstly, adjusting the pH value of the hydrolysate to 5.0 by NaOH and then |
| | adding 10% (v/v) laccase (2.75 U/mL) to the hydrolysate. Secondly, incubating |
| | the hydrolysate at 30° C. for 12 hours and adjusting the pH value to 5.0 again. |
| B2 | Firstly, adjusting the pH value of the hydrolysate to 5.0 by Ca(OH)₂ and then |
| | adding 10% (v/v) laccase (2.75 U/mL) to the hydrolysate. Secondly, incubating |
| | the hydrolysate at 30° C. for 12 hours and adjusting the pH value to 5.0 again. |
| C2 | Firstly, adjusting the pH value of the hydrolysate to 5.0 by ammonia and then |
| | adding 10% (v/v) laccase (2.75 U/mL) to the hydrolysate. Secondly, incubating |
| | the hydrolysate at 30° C. for 12 hours and adjusting the pH value to 5.0 again. |

In some embodiments, after harvesting and/or detoxification, the tensile strength of the bacterial nanocellulose may be measured. In some embodiments, before tensile strength measurement, the bacterial nanocellulose may be soaked in a basic solution such as, for example, a 0.5% to 2% NaOH solution. For example, the NaOH solution may be between about 0.5% and about 0.75%, about 0.75% and about 1.0%, about 1.0% and about 1.25%, about 1.25% and about 1.5%, about 1.5% and about 1.75%, about 1.75% and about 2.0%, or any concentration between these concentrations. The bacterial nanocellulose may also be heated at temperatures between about 60° C. and 100° C., for a time ranging between about 30 minutes and 240 minutes. For example, the bacterial nanocellulose may be heated to about 60° C., about 70° C., about 80° C., about 90° C., about 100° C., or any temperature in between these temperatures. The duration of heating may range, in some embodiments, from between about 30 minutes to about 60 minutes, about 60 minutes to about 90 minutes, about 90 minutes to about 120 minutes, about 120 minutes to about 150 minutes, about 150 minutes to about 180 minutes, about 180 minutes to about 240 minutes, or any time between these ranges. The soaking and heating function, in some embodiments, to remove impurities such as culture medium and trapped bacterial cells. In some embodiments, the bacterial nanocellulose may be subdivided into smaller portions for analysis of tensile strength. In some embodiments the tensile strength of wet bacterial nanocellulose may be measured directly, such as by using a universal testing machine (H5K-S, Hounsfield Test Equipment Ltd, UK) operating at suitable parameters established in the art. Generally, all data for determination of tensile strength are collected under the same conditions. The tensile strength (in megapascal, MPa, or N/mm²) may be calculated, for example, by dividing the tensile force by the area of the cross section of the bacterial nanocellulose tested or using other calculations known to one of skill in the art. The tensile strength, in some embodiments, ranges from about 0.01 MPa to about 0.1 MPa, for example, from about 0.01 MPa to about 0.02 MPa, about 0.02 MPa to about 0.03 MPa, about 0.03 MPa to about 0.04 MPa, about 0.04 MPa to about 0.05 MPa, about 0.05 MPa to about 0.06 MPa, about 0.06 MPa to about 0.07 MPa, about 0.07 MPa to about 0.08 MPa, about 0.08 MPa to about 0.09 MPa, about 0.09 MPa to about 0.1 MPa, or any tensile strength between those listed. Moreover, the bacterial nanocellulose, in some embodiments, may optionally be dried prior to testing tensile strength (or subject it to further processing). In several embodiments, the dry weight of bacterial nanocellulose obtained using the methods and compositions disclosed herein ranges from about 5 g/mL to about 20 g/mL, for example, about 5 g/mL to about 7 g/mL, about 7 g/mL to about 9 g/mL, about 9 g/mL to about 11 g/mL, about 11 g/mL to about 13 g/mL, about 13 g/mL to about 15 g/mL, about 15 g/mL to about 17 g/mL, about 17 g/mL to about 18 g/mL, about 18 g/mL to about 20 g/mL, or any weight between those listed. Greater dry weights are achieved in certain embodiments.

Cellulosic plant fibers such as Sorghum bagasse and sugar cane bagasse have been widely distributed and also widely cultivated in the industry. In particular, as the Sorghum plant can resist aridity and salinity, it can be planted in lean soil. Sorghum and sugar cane contain abundant sugar, minerals and vitamins. Hence, the cellulosic plant fibers derived therefrom can be suitable biomass to produce bacterial nanocellulose. In addition, these cellulosic plant fibers can be beneficial for decreasing the production cost of bacterial nanocellulose and subsequently increasing the scale of industrial manufacture of bacterial nanocellulose.

The cellulosic plant fibers as described in the disclosed embodiments, contain abundant sugar content and are expected to give a higher sugar yield after hydrolyzation. The sugar can then be used as a carbon source in the fermentation media to produce bacterial nanocellulose.

The sweet juice of the cellulosic plant fibers, the hydrolysates, or their mixtures can remarkably improve the yield of bacterial nanocellulose, which can decrease the production cost in large-scale manufacture.

### EXAMPLES

### Example 1—Hydrolysis with Inorganic Acid

Sorghum bagasse is mixed with 4% weight/volume diluted sulfuric acid aqueous solution for about 12 hours in a container. The ratio of solid to liquid is adjusted to approximately 1:15. The hydrolysis reaction is then allowed to proceed at approximately 130° C. for approximately 140 minutes.

### Example 2—High Temperature Hydrolysis with Inorganic Acid

Sorghum bagasse is mixed with 4% weight/volume diluted sulfuric acid aqueous solution for about 12 hours in a container. The ratio of solid to liquid is adjusted to approximately 1:15. The hydrolysis reaction is then allowed to proceed at approximately 200° C. for about 30 minutes.

### Example 3—Low Temperature Hydrolysis with Inorganic Acid

Sorghum bagasse is mixed with 4% weight/volume diluted sulfuric acid aqueous solution for about 12 hours in a container. The ratio of solid to liquid is adjusted to approximately 1:15. The hydrolysis reaction is then allowed to proceed at approximately 80° C. for about 180 minutes.

### Example 4—Hydrolysis with Organic Acid

Sorghum bagasse is mixed with 4% w/v diluted acid acetic acid aqueous solution for about 12 hours in a container. The ratio of solid to liquid is adjusted to approximately 1:15. The hydrolysis reaction is then allowed to proceed at approximately 130° C. for approximately 140 minutes.

### Example 5—High Temperature Hydrolysis with Organic Acid

Sorghum bagasse is mixed with 4% w/v diluted acid acetic acid aqueous solution for about 12 hours in a container. The ratio of solid to liquid is adjusted to approximately 1:15. The hydrolysis reaction is then allowed to proceed at approximately 200° C. for about 30 minutes.

### Example 6—Low Temperature Hydrolysis with Organic Acid

Sorghum bagasse is mixed with 4% w/v diluted acid acetic acid aqueous solution for about 12 hours in a container. The ratio of solid to liquid is adjusted to approximately 1:15. The hydrolysis reaction is then allowed to proceed at approximately 80° C. for about 180 minutes.

### Example 7—Enzymatic Hydrolysis with a Single Saccharification Enzyme

Sorghum bagasse is mixed with 300 U of cellulase in a container. The ratio of solid to liquid is adjusted to approximately 1:20. Reactions are allowed to proceed at approximately 50° C. for about 48 hours.

### Example 8—Enzymatic Hydrolysis with an Elevated Concentration of a Single Saccharification Enzyme

Sorghum bagasse is mixed with 700 U of cellulase in a container. The ratio of solid to liquid is adjusted to approximately 1:20. Reactions are allowed to proceed at approximately 50° C. for about 24 hours.

### Example 9—Low Temperature Enzymatic Hydrolysis with a Single Saccharification Enzyme

Sorghum bagasse is mixed with 300 U of endogluconase in a container. The ratio of solid to liquid is adjusted to approximately 1:20. Reactions are allowed to proceed at approximately 45° C. for about 72 hours.

### Example 10—Elevated Temperature Enzymatic Hydrolysis with a Single Saccharification Enzyme

Sorghum bagasse is mixed with 300 U of endogluconase in a container. The ratio of solid to liquid is adjusted to approximately 1:20. Reactions are allowed to proceed at approximately 55° C. for about 24 hours.

### Example 11—Enzymatic Hydrolysis Using Mixtures of Saccharification Enzymes

Sorghum bagasse is mixed with a total of 500 U of a mixture of saccharification enzymes comprising cellulase, hemicellulose, xylanase, protease, endogluconase, cellobiase, lipase, amylase, glucan glucohydrolase, and glucoamylase in a container. The ratio of solid to liquid is 1:20. Reactions are allowed to proceed at approximately 50° C. for about 48 hours.

### Example 12—Enzymatic Hydrolysis Using Mixtures of Saccharification Enzymes at an Elevated Concentration

Sorghum bagasse is mixed with a total of 700 U of a mixture of saccharification enzymes comprising cellulase, hemicellulose, xylanase, protease, endogluconase, cellobiase, lipase, amylase, glucan glucohydrolase, and glucoamylase in a container. The ratio of solid to liquid is 1:20. Reactions are allowed to proceed at approximately 50° C. for about 24 hours.

### Example 13—Low Temperature Enzymatic Hydrolysis Using Mixtures of Saccharification Enzymes

Sorghum bagasse is mixed with a total of 500 U of a mixture of saccharification enzymes comprising cellulase, hemicellulose, xylanase, protease, endogluconase, cellobiase, lipase, amylase, glucan glucohydrolase, and glucoamylase in a container. The ratio of solid to liquid is 1:20. Reactions are allowed to proceed at approximately 45° C. for about 72 hours.

### Example 14—Elevated Temperature Enzymatic Hydrolysis Using Mixtures of Saccharification Enzymes

Sorghum bagasse is mixed with a total of 500 U of a mixture of saccharification enzymes comprising cellulase, hemicellulose, xylanase, protease, endogluconase, cellobiase, lipase, amylase, glucan glucohydrolase, and glucoamylase in a container. The ratio of solid to liquid is 1:20. Reactions are allowed to proceed at approximately 55° C. for about 24 hours.

### Example 15—Detoxification Treatment Using Sodium Hydroxide and Activated Charcoal

The pH value of sorghum bagasse acid hydrolysate is adjusted to pH 10.0 by addition of NaOH. That mixture is incubated at 30° C. for 12 hours, and the pH is adjusted to pH 5.0 H₂SO₄. Thereafter, 3% (w/v) activated charcoal is added into the hydrolysate and mixed for 10 minutes. Finally, the activated charcoal is removed from the hydrolysate by centrifugation and the pH is adjusted again to pH 5.0 using H₂SO₄.

### Example 16—Additional Detoxification Treatment Using Sodium Hydroxide and Activated Charcoal

The pH value of sorghum bagasse acid hydrolysate is adjusted to pH 10.0 by addition of NaOH. That mixture is incubated at 30° C. for 12 hours, and the pH is adjusted to pH 5.0 using H₂SO₄. Thereafter, 3% (w/v) activated charcoal is added into the hydrolysate and mixed for 10 minutes. Finally, the activated charcoal is removed from the hydrolysate by filtration and the pH is adjusted again to pH 5.0 using H₂SO₄.

### Example 17—Detoxification Treatment Using Calcium Hydroxide and Activated Charcoal

The pH value of sorghum bagasse acid hydrolysate is adjusted to pH 10.0 by addition of Ca(OH)₂ and that mixture is incubated at 30° C. for 12 hours. Thereafter the pH is adjusted to pH 5.0 using H₂SO₄. Activated charcoal (2% (w/v)) is added into the hydrolysate and mixed them for 5 minutes. The activated charcoal is removed from the hydrolysate using centrifugation and the pH value is adjusted to 5.0 again using H₂SO₄.

### Example 18—Additional Detoxification Treatment Using Calcium Hydroxide and Activated Charcoal

The pH value of sorghum bagasse acid hydrolysate is adjusted to pH 10.0 by addition of Ca(OH)₂ and that mixture is incubated at 30° C. for 12 hours. Thereafter the pH is adjusted to pH 5.0 using H₂SO₄. 2% (w/v) activated charcoal is added into the hydrolysate and mixed them for 5 minutes. The activated charcoal is removed from the hydrolysate using filtration and the pH value is adjusted to 5.0 again using H₂SO₄.

### Example 19—Detoxification Treatment Using Ammonia and Activated Charcoal

The pH value of sorghum bagasse acid hydrolysate is adjusted to pH 10.0 by addition of ammonia and that mixture is incubated at 30° C. for 12 hours. Thereafter, the pH value is adjusted to pH 5.0 using H₂SO₄. Secondly, adding 2% (w/v) activated charcoal into the hydrolysate and mixing them for 5 minutes. Finally, the activated charcoal is removed from the hydrolysate using centrifugation and the pH value is adjusted to pH 5.0 again using H₂SO₄.

### Example 20—Additional Detoxification Treatment Using Ammonia and Activated Charcoal

The pH value of sorghum bagasse acid hydrolysate is adjusted to pH 10.0 by addition of ammonia and that mixture is incubated at 30° C. for 12 hours. Thereafter, the pH value is adjusted to pH 5.0 using H₂SO₄. Secondly, adding 2% (w/v) activated charcoal into the hydrolysate and mixing them for 5 minutes. Finally, the activated charcoal is removed from the hydrolysate using filtration and the pH value is adjusted to pH 5.0 again using H₂SO₄.

### Example 21—Detoxification Treatment Using Sodium Hydroxide and Laccase

The pH value of sorghum bagasse acid hydrolysate is adjusted to pH 5.0 by addition of NaOH. Thereafter, laccase (2.75 U/mL) is added to the pH adjusted hydrolysate to a 10% (v/v) concentration. That mixture is incubated at 30° C. for 12 hours, and then the pH is adjusted to 5.0 by using H₂SO₄.

### Example 22—Detoxification Treatment Using Calcium Hydroxide and Laccase

The pH value of sorghum bagasse acid hydrolysate is adjusted to pH 5.0 by addition of Ca(OH)₂. Thereafter, laccase (2.75 U/mL) is added to the pH adjusted hydrolysate to a 10% (v/v) concentration. That mixture is incubated at 30° C. for 12 hours, and then the pH is adjusted to 5.0 by using H₂SO₄.

### Example 23—Detoxification Treatment Using Ammonia and Laccase

The pH value of sorghum bagasse acid hydrolysate is adjusted to pH 5.0 by addition of ammonia. Thereafter, laccase (2.75 U/mL) is added to the pH adjusted hydrolysate to a 10% (v/v) concentration. That mixture is incubated at 30° C. for 12 hours, and then the pH is adjusted to 5.0 by using H₂SO₄.

### Example 24—Direct Production of Culture Medium Using Crushed Sorghum

After crushing and hydrolyzing sweet sorghum, the liquid portion of the hydrolysate is separated from the solid portion (e.g., slag), is sterilized, and then is directly used as a culture medium for growth of nanocellulose-producing bacteria.

### Example 25—Production of Culture Medium Using Crushed Sorghum

Sweet sorghum hydrolysate (whether generated by enzymatic or acid hydrolysis) is supplemented with 2% organic nitrogen (wt %) in the form of yeast extract and 0.3% calcium. The resultant mixture is autoclaved at 110° C. for 30 min and used as a culture medium for growth of nanocellulose-producing bacteria.

### Example 26—Production of Culture Medium Using Filtrate of Crushed Sorghum

Detoxified acid hydrolysate of sweet sorghum bagasse is combined with (w/v) 0.3% peptone, 0.5% yeast extract, and 2.5% glucose. The resultant mixture is sterilized with a sterile filter and used as a culture medium for growth of nanocellulose-producing bacteria.

### Example 27—Bacterial Nanocellulose Production

Nanocellulose producing bacteria are inoculated into a sterilized culture media until the inoculum is present at 5% (v/v). The culture is incubated at 37° C. in an oscillating incubator (set at about 200 rpm) for 10 days. Bacterial nanocellulose is harvested by filtration. Thereafter, the bacterial nanocellulose is washed with deionized water and the dry weight of the nanocellulose is determined after drying at 105° C.±0.5° C. for 24 hours. The resultant dry weight of the bacterial nanocellulose is between about 8 g/mL and about 18 g/mL.

### Example 28—Tensile Strength Measurement

Nanocellulose producing bacteria were inoculated into aliquots of sterilized culture seed media comprising (w/v) 0.3% peptone, 0.5% yeast extract, and 2.5% glucose. Bagasse hydrolysate having a reducing sugar concentration of 25-200 g/L was added to each aliquot (to generate a bacterial nanocellulose production media) until the inoculum was present at 5% (v/v). Each culture was incubated at 30° C. in a static incubator for approximately 10 days. Bacterial nanocellulose was harvested. Thereafter, the bacterial nanocellulose pellicle was washed with deionized water and the dry weight of the nanocellulose was determined after drying at 105° C.±0.5° C. for 24 hours. The resultant dry weight of the bacterial nanocellulose averaged between about 8 g/mL and about 18 g/mL.

Before tensile strength measurement, the bacterial nanocellulose pellicle was soaked in 1% NaOH solution and heated at 80° C. for 120 minutes to remove impurities such as culture medium and trapped bacterial cells. The bacterial nanocellulose pellicle was cut into 40 mm long and 10 mm wide strips for analysis of tensile strength. The tensile strength of wet BNC was measured by using a universal testing machine (H5K-S, Hounsfield Test Equipment Ltd, UK) operating at a crosshead speed of 50 mm/minutes. All data for determination of tensile strength were collected under the same conditions. The tensile strength (in megapascal, MPa, or N/mm²) was calculated by dividing the tensile force by the area of the cross section of the BNC strips. Each test was performed by using 10 samples and mean values of the strength of BNC are given. The strength of BNC averaged 0.03-0.06 Mpa.

With respect to the use of substantially any plural and/or singular terms herein, those having skill in the art can translate from the plural to the singular and/or from the singular to volume of wastewater can be received in the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A method of making bacterial nanocellulose, the method comprising:
contacting a cellulosic plant fiber with a catalyst selected from an acid catalyst, an enzymatic catalyst or a combination thereof to form a reaction mixture;
subjecting the reaction mixture to hydrolyze at least a portion of the cellulosic plant fiber into a hydrolysate;
forming a culture medium with the hydrolysate;
inoculating the culture medium with at least one bacterium; and
incubating the culture medium and the bacteria to produce the bacterial nanocellulose.

2. The method of claim 1, further comprising separating any unhydrolyzed cellulosic plant fiber from the hydrolysate and adding the unhydrolyzed cellulosic plant fiber to the reaction mixture.

3.-4. (canceled)

5. The method of claim 1, further comprising fragmenting the cellulosic plant fiber before contacting with the catalyst, wherein the cellulosic plant fiber has an average particle size of about 250 μm to about 420 μm after the fragmenting step.

6. (canceled)

7. The method of claim 5, further comprising crushing a cellulosic plant to form the cellulosic plant fiber and a juice before the fragmenting step and adding the juice to the hydrolysate when forming the culture medium, wherein the cellulosic plant fiber is Sorghum bagasse, sugar cane bagasse, or both.

8.-10. (canceled)

11. The method of claim 1, wherein forming the culture medium comprises adding at least one nitrogen source, at least one trace element, or both to the hydrolysate.

12. The method of claim 11, wherein the nitrogen source comprises organic nitrogen, and wherein the organic nitrogen source is peptone, yeast extract, tryptone, or a combination thereof.

13. (canceled)

14. The method of claim 11, wherein the nitrogen source comprises inorganic nitrogen, and wherein the inorganic nitrogen source is ammonium sulfate, ammonium chloride, or both.

15. (canceled)

16. The method of claim 11, wherein the trace element comprises calcium, magnesium, or both.

17. The method of claim 11, wherein the nitrogen source is present in the culture medium at a concentration of about 0.1% to about 1% by weight, and the hydrolysate is present in the culture medium at a concentration of about 1% to about 10% by weight, and the trace element is present in the culture medium at a concentration of about 0.1% to about 0.5% by weight.

18. The method of claim 1, wherein the bacteria is Gluconacetobacter xylinus, Gluconacetobacter hansenii, Gluconobacter oxydans, Rhizobium sp., Sarcina sp., Pseudomounas sp., Achromobacter sp., Alcaligenes sp., Aerobacter sp., Azotobacter sp., Agrobacterium sp., Seudomonas cepacia, Campylobacter jejuni, or a combination thereof, and wherein the bacteria is present in the culture medium at a concentration of about 3% to about 15% by volume.

19. (canceled)

20. The method of claim 1, wherein the hydrolysate has a reducing sugar concentration of about 5 g/L to about 200 g/L.

21. The method of claim 1, wherein the incubating step comprises: incubating the culture medium and the bacteria at a temperature of about 20° C. to about 37° C. for about 1 day to about 30 days.

22.-23. (canceled)

24. The method of claim 1, wherein the incubating step comprises: incubating the culture medium and the bacteria in a shaking incubator that rotates at a speed of about 5 rpm to about 300 rpm.

25. The method of claim 1, further comprising harvesting the bacterial nanocellulose from the culture medium.

26. The method of claim 25, further comprising contacting the bacterial nanocellulose with a base under conditions to remove residual bacteria and culture medium, wherein contacting the bacterial nanocellulose with the base comprises heating at about 70° C. to about 120° C. for about 90 minutes to about 150 minutes, and wherein the base is in aqueous form having a concentration of about 0.5% to about 8% by weight.

27.-30. (canceled)

31. The method of claim 1, wherein the subjecting step comprises: subjecting the reaction mixture to a temperature of about 25° C. to about 200° C. for about 10 minutes to about 48 hours.

32. The method of claim 1, wherein contacting the cellulosic plant fiber with the catalyst comprises: contacting an aqueous form of the catalyst with the cellulosic plant fiber, wherein the cellulosic plant fiber and the aqueous form of the catalyst are present in the reaction mixture at a ratio of about 1:5 to about 1:30 by weight to volume (w/v).

33. (canceled)

34. The method of claim 1, wherein the catalyst is an acid catalyst and wherein the acid catalyst is an aqueous acid solution having a concentration of about 0.3% to about 10% w/v.

35.-36. (canceled)

37. The method of claim 1, wherein contacting the cellulosic plant fiber with the acid catalyst comprises: contacting the cellulosic plant fiber with the acid catalyst for about 12 hours to about 24 hours.

38. The method of claim 1, wherein the subjecting step comprises: subjecting the reaction mixture to a temperature of about 25° C. to about 200° C. for about 10 minutes to about 90 minutes.

39. The method of claim 1, wherein the enzymatic catalyst is a saccharification enzyme selected from cellulase, hemicellulose, xylanase, protease, lipase, amylase, glucan glucohydrolase, glucoamylase, and a combination thereof, and wherein the enzymatic catalyst has an enzyme unit of about 1 U to about 700 U.

40.-42. (canceled)

43. The method of claim 1, wherein the subjecting step comprises: subjecting the reaction mixture to a temperature of about 25° C. to about 90° C. for about 30 minutes to about 48 hours.

44. The method of claim 1, further comprising detoxifying the hydrolysate after the subjecting step, and before inoculating the culture medium with the at least one bacteria, wherein the detoxifying the hydrolysate comprises:
adjusting a pH value of the hydrolysate to an alkaline pH with a base, wherein the alkaline pH is about pH 10;
incubating the hydrolysate; adjusting the pH value of the hydrolysate to an acidic pH with an acid, wherein the acidic pH is about pH 5;
contacting the hydrolysate with activated carbon; and
separating the activated carbon from the hydrolysate.

45. (canceled)

46. The method of claim 44, further comprising adjusting the pH of the hydrolysate to the acidic pH after separating the activated carbon from the hydrolysate.

47.-50. (canceled)

51. The method of claim 44, wherein incubating the hydrolysate comprises incubating at a temperature of about 20° C. to about 50° C. for about 6 hours to about 24 hours.

52.-54. (canceled)

55. The method of claim 44, wherein detoxifying the hydrolysate comprises:
adjusting a pH value of the hydrolysate to an alkaline pH with a base, or to an acidic pH with an acid; contacting the hydrolysate with an enzyme wherein the enzyme has an enzyme unit concentration of about 2.75 U/ml; and incubating the hydrolysate and further adjusting the pH value of the hydrolysate to the alkaline pH or to the acidic pH after incubating.

56.-60. (canceled)

61. The method of claim 55, wherein the enzyme is laccase or peroxidase with H₂O₂.

62.-64. (canceled)

65. The method of claim 55, wherein the enzyme is present in the hydrolysate in a concentration of about 1% to about 20% by volume.

66. The method of claim 55, wherein incubating the hydrolysate comprises incubating at a temperature of about 20° C. to 90° C. for about 1 hour to about 48 hours.

67.-69. (canceled)

70. A method of making a culture medium, the method comprising:
contacting a cellulosic plant fiber with a catalyst selected from an acid catalyst, an enzymatic catalyst or a combination thereof, to form a reaction mixture;
subjecting the reaction mixture to hydrolyze at least a portion of the cellulosic plant fiber into a hydrolysate; and
forming the culture medium with the hydrolysate.

71. (canceled)

72. The method of claim 70, further comprising: crushing a cellulosic plant to form the cellulosic plant fiber and a juice, fragmenting the cellulosic plant fiber before contacting with the catalyst wherein the cellulosic plant fiber is Sorghum bagasse, sugar cane bagasse, or both and wherein the cellulosic plant fiber has an average particle size of about 250 μm to about 420 μm after the fragmenting step, and adding the juice to the hydrolysate when forming the culture medium.

73.-77. (canceled)

78. The method of claim 70, wherein the hydrolysate has a reducing sugar concentration of about 5 g/L to about 200 g/L.

79. (canceled)

80. The method of claim 70, wherein contacting the cellulosic plant fiber with the catalyst comprises: contacting an aqueous form of the catalyst with the cellulosic plant fiber, wherein the cellulosic plant fiber and the aqueous form of the catalyst are present in the reaction mixture at a ratio of about 1:5 to about 1:30 by weight to volume (w/v).

81. (canceled)

82. The method of claim 70, wherein the catalyst is an aqueous acid solution having a concentration of about 0.3% w/v to about 10% w/v.

83.-84. (canceled)

85. The method of claim 70, wherein contacting the cellulosic plant fiber with the acid catalyst comprises: contacting the cellulosic plant fiber with the acid catalyst for about 12 hours to about 24 hours.

86. The method of claim 70, wherein the subjecting step comprises: subjecting the reaction mixture to a temperature of about 25° C. to about 200° C. for about 10 minutes to about 90 minutes, when the cellulosic plant fiber is contacted with the acid catalyst.

87. The method of claim 70, wherein the enzymatic catalyst is a saccharification enzyme selected from cellulase, hemicellulose, xylanase, protease, lipase, amylase, glucan glucohydrolase, glucoamylase, and a combination thereof, and wherein the enzymatic catalyst has an enzyme unit of about 1 U to about 700 U.

88.-89. (canceled)

90. The method of claim 70, wherein the subjecting step comprises: subjecting the reaction mixture to a temperature of about 25° C. to about 90° C. for about 30 minutes to about 48 hours, when the cellulosic plant fiber is contacted with the enzymatic catalyst.

91. The method of claim 70, further comprising detoxifying the hydrolysate after the subjecting step, wherein detoxifying the hydrolysate comprises: adjusting a pH value of the hydrolysate to an alkaline pH with a base; incubating the hydrolysate; adjusting the pH value of the hydrolysate to an acidic pH with an acid; contacting the hydrolysate with activated carbon; and separating the activated carbon from the hydrolysate.

92.-98. (canceled)

99. The method of claim 91, wherein detoxifying the hydrolysate comprises: adjusting a pH value of the hydrolysate to an alkaline pH with a base, or to an acidic pH with an acid; contacting the hydrolysate with an enzyme wherein the enzyme has an enzyme unit concentration of about 1 U/ml to about 50 U/ml; and incubating the hydrolysate.

100.-102. (canceled)

103. The method of claim 99, wherein the enzyme is laccase or peroxidase with H₂O₂.

104.-105. (canceled)

106. The method of claim 99, wherein the enzyme is present in the hydrolysate at a concentration of about 1-20% by volume.

107. The method of claim 99, wherein incubating the hydrolysate comprises incubating at a temperature of about 20° C. to 90° C. for about 1 hour to about 48 hours.

108.-120. (canceled)

121. A culture medium comprising: a hydrolysate prepared by contacting a cellulosic plant fiber, wherein the cellulosic plant fiber is Sorghum bagasse, sugar cane bagasse, or both, with a catalyst selected from an acid catalyst, an enzymatic catalyst or a combination thereof, to form a reaction mixture; and subjecting the reaction mixture to hydrolyze at least a portion of the cellulosic plant fiber into the hydrolysate, wherein the hydrolysate has a reducing sugar concentration of about 5 g/L to about 200 g/L.

122. The culture medium of claim 121, further comprising at least one nitrogen source, at least one trace element, or both, wherein the nitrogen source is present in the culture medium at a concentration of about 0.1% to about 1% by weight, and the hydrolysate is present in the culture medium at a concentration of about 1% to about 10% by weight, and the trace element is present in the culture medium at a concentration of about 0.1% to about 0.5% by weight.

123. (canceled)

124. The culture medium of claim 122, wherein the nitrogen source comprises organic nitrogen, wherein the organic nitrogen is peptone, yeast extract, tryptone or a combination thereof.

125. (canceled)

126. The culture medium of claim 122, wherein the nitrogen source comprises inorganic nitrogen, wherein the inorganic nitrogen is ammonium sulfate, ammonium chloride or both.

127. (canceled)

128. The culture medium of claim 122, wherein the trace element comprises calcium, magnesium or both.

129.-140. (canceled)
